(19) United States
(12) Patent Application Publication
(10) Pub. No.: US 2004/0115755 A1
(43) Pub. Date: Jun. 17, 2004

(54) Method and apparatus for detecting response to damage and diagnostic method therefor

(76) Inventors: Ronald Robert David Croy, Durham (GB); Steven Michael Johnson, Durham (GB); Sean James Doherty, Durham (GB)

Correspondence Address: JACOBSON HOLMAN PLLC, 400 SEVENTH STREET N.W., SUITE 600, WASHINGTON, DC, 20004, US

(21) Appl. No.: 10/474,036

(22) PCT Filed: Apr. 12, 2002
(86) PCT No.: PCT/GB02/01631

(30) Foreign Application Priority Data
Apr. 12, 2001 (GB) O109150.3

(51) Int. Cl. C12Q 1/26

(52) U.S. Cl. 435/25

(57) ABSTRACT

Method for detecting response to damage or disease in a plant or crop comprising detecting Active Oxygen Species (AOS), in a quantitative assay; diagnostic reagent comprising an extract of damaged or diseased plant or crop tissue, or active component thereof, or synthetic equivalent or derivative thereof; use of the method or reagent in detecting response to damage or disease in a plant or crop selected from potato (seed, processor or consumer), apple, lychee, pear, apricot, peach, orange (juice), banana, leaf or stem browning in cut flowers, lettuce, pomegranate, grape, mushroom, logan fruit, loquat fruit, blueberry, carambola, dog rose, rambutan, coconut, avocado, plantain, pineapple, walnut, artichoke, sugarbeet and onion; apparatus and kit therefor; and associated methods for modifying and selecting varieties, growth or environmental conditions of a plant or crop; and use therein of a computer programmed with data relating to susceptibility of a plurality of plants and crops to damage or disease.

The present invention relates to a method for detecting a response to damage or disease in a plant or crop and for diagnosing the susceptibility of a plant or crop to respond to damage or disease, an apparatus and diagnostic tool for use in the method, a method for improving the growth or handling conditions of a plant or crop to minimise the risk of damage or disease and for selecting a plant or crop which is diagnosed as having minimal risk of damage or disease under predetermined growing or handling conditions, a computer programmed with data relating to damage or disease susceptibility for a given plant or crop and the use thereof to select a given plant or crop suited to predetermined growth or handling conditions.

Bruising or softening occurs in susceptible crops as the result of impact damage, most frequently during harvesting, transport and handling. This can result in loss of the affected item or can lead to local or wide spread rotting of neighbouring items or a whole crop, by providing an entry point for pathogenic disease. It is known that susceptibility to bruising or softening is a variable phenomenon in certain crops, for example in the case of black spot bruising in potato tubers, this is affected by variety, growth and environmental conditions. Adjustment of harvesting and handling machinery or manual procedures can reduce impact and bruise damage but this is only possible if prediction of the level of susceptibility of the crop allows growers to anticipate problems before harvesting commences. The ability to rapidly and accurately predict the bruising status of a plant or crop would allow growers to optimise harvester settings and handle susceptible crops carefully and therefore maximise resources.

The pigments that characterise black spot bruising in potato tubers arise through complex chemical events involving tuber factors and oxygen, and produce initially red-brown chemical intermediates, and lead ultimately to the formation of the characteristic blue-black melanin pigments.

Assessment of tuber susceptibility to black spot bruising has traditionally been based on direct visualisation of tuber dis-colouration following artificial impact. This is a prolonged assay involving incubation of tubers for up to 48 hours and requires specialised equipment and trained staff. Attempts have been made to find alternative tuber factors or properties which reliably indicate the susceptibility of tubers to bruising. These include chemical factors such as tyrosine, enzymes and cellular respiration or physical properties such as specific gravity, water content, dry matter content, tuber turgor and “firmness”. None of these has proved to be reliable and subsequent data has failed to confirm a reproducible correlation with black spot bruising susceptibility in all situations.

Synthesis of AOS such as hydrogen peroxide and superoxide is a primary response of plant cells to a pathogen attack or to treatment with chemical elicitors but few reports have described this response to a mechanical stimulus. Yahraus et al. (Evidence for a mechanically induced oxidative burst. Plant Physiol 109: 1259-1266-1995) simulated mechanical stress in soybean cell suspension cultures by hypo-osmotic media and demonstrated hydrogen peroxide synthesis within minutes. They also imposed direct mechanical stress by exerting physical pressure on the cells under a microscope slide and were able to demonstrate peroxide production histochemically after 3 minutes. Legendre et al. (Characterisation of the oligogalacturonide-induced oxidative burst in cultured soybean (Glycine max) cell. Plant Physiol 102: 233-240-1993) and Cazalé et al. (Oxidative burst and hypoosmotic stress in tobacco cell suspensions. Plant Physiol 116: 659-669-1998, MAP kinase activation by hypoosmotic stress of tobacco cell suspensions: towards the oxidative burst response? Plant J 19: 297-307-1999) used mixing of cell suspensions to impose mechanical stress and showed an oxidative burst of peroxide synthesis. However none of these studies drew any quantitative relations with damage incurred.

Generation of superoxide radicals as a consequence of impact damage was reported by Baker and Orlandi (Active oxygen in plant pathogenesis. Annu Rev Phytopathol 33: 299-321-1995) as a biphasic oxidative burst in which cultured cells treated with elicitor displayed two peaks of hydrogen peroxide generation over a period of 6-8 hours. Phase 1 was suggested to be a non-specific biological response to stress while phase 2 was determined to be a specific interaction between the pathogen hrp complex and the plant cell receptors leading to hypersensitive cell death. Again these studies neither disclose nor suggest the use of superoxide measurement as a quantitative assay or indicator of susceptibility to bruising.

Accordingly there is a need for an improved method for assessing potato tuber susceptibility to black spot bruising and indeed for assessing susceptibility of plants and crops in general to bruising or softening. It is therefore an object of the present invention to determine factors which are correlated with bruise susceptibility and which are capable of being conveniently measured.

We have investigated the early responses of tuber tissues to impact damage. We have surprisingly found that the ability to generate free radicals vary significantly between different potato varieties and in fact the variation in the ability to produce free radicals correlates exactly with the degree of susceptibility of varieties to bruising. However these species are highly reactive molecules not normally present in large amounts in tissues and it was therefore even more surprising to find that these species are synthesised in sufficient levels to be detected.

Accordingly in the broadest aspect of the invention there is provided a method for detecting response to damage or disease in a plant or crop comprising detecting Active Oxygen Species (AOS), in a quantitative assay. Preferably the method comprises detecting the oxygen free radicals.

The terms damage and disease are used to indicate different origins of AOS generation but may be used interchangeably in some instances, for example where the origin involves one event leading to heightened susceptibility to the other or AOS generation of indeterminate event. The term damage may therefore be used generically or specifically herein.

Specific reference herein to damage is to any mechanical damage such as injury, wounding, bruising, stress or trauma and the like incurred as a result of strike or impact, (bruising), force or increased pressure, vibration, shock, slicing, stabbing, penetration, sudden or prolonged temperature depression or elevation and the like.

Reference herein to disease is to any pathogenic disease which causes cellular disruption ranging from slight leaking of cell contents to full disruption, such as fungal or like disease.

Reference herein to bruise or bruising may be to the action of bruising a plant or crop or to the phenomenon resulting from impact or mechanical damage, including not only the characteristic coloration but also other responses such as softening and cell death. The phenomenon of bruising is intended to include discoloration resulting from mechanical damage and therefore indicates not just the physical injury due to impact but necessarily the resulting biochemical and chemical events leading to a visible discoloration of the impacted tissue. While this happens in several fruits and vegetables it is likely that not all crops respond in this way to impact or other mechanical injury eg. do not respond by production of pigments (eg. by softening, cell death), and discoloration is absent. Accordingly the invention includes detection of susceptibility in crops whereby discoloration does not take place.

The chemical events causing crop bruising are to some degree known. Without being limited to this theory however we have evidence that impact damage causes cellular disruption and leads to de-compartmentalisation of the enzyme polyphenol oxidase which mixes with monophenolic substrates such as tyrosine. Oxygen dependent reactions produce initially the red-brown dihydroxyphenylalanine intermediates and lead ultimately to the formation of the characteristic blue-black melanin pigments which polymerise to water insoluble complexes. Evidence points to the involvement of bound tuber proteins in these complexes. We believe that AOS are generated simultaneously or subsequently to complex formation, possibly as a result of the breakdown of cells, enabling contact of enzyme and amino acid tyrosine.

In a surprising advantage of our invention therefore the method comprises detecting AOS outside the cell from a tissue sample or by in situ probe.

It may be that there is actually a causal link between AOS production and bruising. The series of events might take place as follows—impact causes i) activation of AOS production (via receptors etc) presumably in viable cells (ie non-disrupted cells) ii) cellular disruption causing mixing of PPO enzyme and tyrosine; iii) AOS promotes the reactions leading to melanin synthesis (some evidence points to activation of PPO and/or the preferential use of free radicals in some of the reactions). Evidence from animal melanin pigment synthesis indicates that the reaction intermediates including the final melanin pigments are “free radical scavengers” that can consume free radicals. Other evidence indicates that certain reactions between tyrosine and melanin may in fact regenerate free radicals (AOS). How the overall reaction is balanced is unknown and is probably very complex. In a particular advantage of the present invention by specifically inhibiting the free radical production we also inhibit bruise pigment production.

A similar result is found to take place on exposure of cells to extract from fungal pathogen, whereby they produce AOS which again are detected outside the cell. Accordingly the invention derives from the finding that plants and crops are characterised by an extra-cellular factor which is both correlated with damage or disease susceptibility and which serves as a signal or a marker of damage or disease which moreover may be conveniently measured.

Reference herein to Active Oxygen Species is to non molecular Oxygen Species, in particular to one or more oxygen free radicals, more particularly selected from superoxide (.O₂⁻), hydroperoxyl radical .O₂H, nitric oxide radical NO., peroxynitrite radical ONOO⁻, hydroxyl radical .OH and derivatives thereof.

It is a particular advantage of the invention that because these species are highly reactive and short lived (of the order of milli seconds) their detection is sensitive, rapid and specific. This makes them ideally suited for use in a diagnostic method and kit for field use. It is a further advantage of the invention that the generation of AOS is a rapid response compared with the bruising response. AOS can be detected within one hour following impact in tubers and the levels of AOS generated progressively increase during the following five to six hours, in comparison with bruising itself which may only develop after 48 hours. It is a further surprising advantage of the invention that the response is linear, whereby a highly bruise susceptible crop shows a high level of AOS generation while a bruise resistant crop shows only a very low level of AOS synthesis.

Preferably the method is for detecting one or more radical types which is easy, rapid and accurate to detect quantitatively. More preferably the method is for detecting superoxide radical.

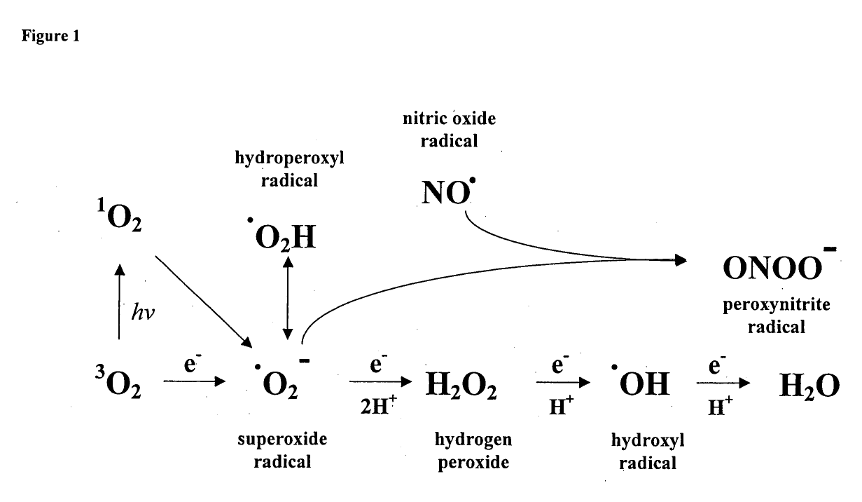

It is known, as illustrated in FIG. 1, that Molecular Oxygen Species form Active Oxygen Species which are inter-related, whereby individual species are short lived. We have found that the “oxidative burst” generated by plants or crops as a response to damage or disease provides AOS at a synthesis rate far exceeding their half life, whereby they are detectable. More specifically we have found that since individual species and specifically superoxide radical are generated in a bi-phasic response providing first and second peak concentrations of AOS at a time t from damage or disease they can be readily detected and comparative studies made giving an indication of damage or disease status.

Preferably the method of the invention comprises detecting AOS in a second peak of bi-phasic response. The observation that a second burst arises in mechanically stressed tissues in the absence of elicitors or pathogens suggests the possible role of a cell receptor for detecting mechanical stimuli and an activation process. This is the first demonstration of what may be a specific response to mechanical stress.

The method may be for detecting whether damage or disease has occurred, in which case the method simply comprises conducting the assay on the suspected site of damage or disease.

Alternatively and preferably the method is for diagnosing the susceptibility of a given plant or crop to respond to damage or disease, for example as a reflection of the general “health” thereof, influenced by growth conditions, cultivar, and the like.

Preferably therefore the method comprises in a first stage exposing the plant or crop to damage or disease and in a second stage detecting AOS in a quantitative assay, at a time t after exposure. Preferably time t is up to 7 hours after exposure, preferably 0.5 to 6 hours after exposure, more preferably 0.5 to 2 hours or 4.5 to 6 hours after exposure.

We have surprisingly found, as hereinbefore mentioned, that the ability to generate free radicals varies significantly between different varieties and crops within a plant or crop species. Advantageously therefore the method of the invention is a method for diagnosing the susceptibility of a plant or crop to respond in an adverse manner to damage or disease. In bruise resistant crops or varieties the AOS synthesis is only slightly greater than background (non damaged or diseased) levels even after a time t of up to 5 hours.

Exposure to damage or disease according to the method of the invention for the purpose of inducing and detecting AOS generation may be direct or indirect.

Direct exposure to damage or disease for the purpose of inducing and detecting AOS generation is to any mechanical damage, for example by impact, pressure, vibration, shock etc., and is preferably a representative controlled impact energy, which serves to represent response to any nature of mechanical damage, or to any disease substances or organisms, fungus, bacteria, virus or the like, for example by contact, injection or otherwise contamination.

Indirect exposure to damage or disease for the purpose of inducing and detecting AOS generation includes exposure to damaged or diseased tissue or components thereof, or subjecting to conditions inducing tissue or cell damage or disease, through swelling, humidity, freezing and the like.

Damaged or diseased tissue may be of the same or different plant or crop species or variety, and is preferably the same species, but may be of a different variety which is suited for using as a control exposure. Particularly useful for AOS generation in tuber is indirect damage or disease exposure involving the application of part or whole of an extract prepared from impacted or diseased tuber tissue.

Preferably the stage of inducing damage directly in a plant or crop or preparing damaged tissue for extract and indirect exposure of a plant or crop comprises subjecting the plant or crop to impact at the site to be assayed as hereinbefore defined at suitable intensity of impact in the energy (or work) range of 0.01 to 1.5 Joules, corresponding to a force range of 0.03 Newtons to 5 Newtons. Impact may be by directional (vertical, horizontal etc.) or non-directional means, suitably to confer steady velocity or accelerating impact (gravitational or otherwise) as appropriate, to model a given damage or disease condition. In the case of potato tubers and similar hard tissue or hard skinned plants or crops a suitable intensity is in the range 0.5-1 joules, more preferably 0.6-0.75 J, and for soft plants or crops a substantially reduced energy ascertained on a species by species basis. A suitable intensity may be determined for any given plant or fruit, such as to induce an impact site of approximately 5-10 mm³. This may simply be determined by experiment by measuring the discoloured or bruised site by means of trial impact with a range of energies.

Preferably the stage of indirectly exposing to damage comprises contacting with part or the whole of an extract prepared from impacted tissue as hereinbefore defined. We have surprisingly found that indirect exposure as hereinbefore defined confers enhanced sensitivity and speed of detection. Exposing to extract modifies the biphasic response both in manner to reduce the period for optimal detection of AOS from the order of 4 to 5 hours from exposure to impact to the order of 1 to 2 hours from exposure to extract, moreover the response curve is of sufficient duration to ensure greater reproducibility and reliability of assay, carried out in a period of from 1 to 2 hours after exposure. Preferably therefore the method of the invention comprises exposing the plant or crop to indirect damage or disease, more preferably by contact with a damaged or diseased tissue extract, and in a second stage detecting AOS in a quantitative assay, at a time t after exposure, where t is in the range substantially 1 to 2 hours.

Preferably an extract from tissues exposed to impact comprises excised tissues homogenised and centrifuged to give a clarified soluble extract. The extract may be further treated to isolate a particular component of the extract or to add additional components such as diffusion enhancers, skin digestants and the like.

Alternatively an extract may comprise synthetically prepared equivalents of the damage tissue extract, or derivatives or analogues thereof. Identification of the active component(s) of the tissue extract may allow this to be provided as a diagnostic reagent for use as hereinbefore defined in the present invention.

In a further aspect of the invention there is therefore provided a diagnostic reagent comprising an extract of damaged or diseased plant or crop tissue, or active component thereof, or synthetic equivalent or derivative thereof. The reagent may comprise additional components useful for aiding penetration or contact with a sample to be diagnosed. The reagent preferably comprises extract or component(s) in effective diagnostic amount. The reagent preferably comprises natural or synthetic extract of the same or different crop to that to be diagnosed, and preferably of a different plant or crop which is found to have high diagnostic value, preferably a tuber.

Quantitative assay according to the method of the invention may be carried out by any known means using a suitable control to subtract background AOS levels, for example by detecting AOS in a corresponding undamaged or diseased plant or crop sample, in a different region of the same plant or crop sample which is undamaged or diseased, or in the same plant or crop sample prior to exposure to damage or disease. Subtraction of background non-damaged or diseased AOS levels may be manual or automatic, giving a calculated or simultaneous result, subsequent to or simultaneous with AOS detection.

A positive response detected by the method of the invention is indicative of damage to the plant or crop which affect the appearance, devaluing the crop, and ultimately may result in bruising, shatter bruising (openings in fabric of the tissue), discolouration, soft rot, pigmentation or the like at any time during harvesting, storage, or otherwise shortening the life of the harvested plant or crop.

Suitably quantitative assay for AOS is chemical or electrochemical and gives a visual response, such as a colour or electronic signal response which may be graded by intensity of colour or magnitude of electronic signal.

Preferably chemical assay is by exposing to the plant or crop to be assayed a chemical marker which is known to undergo visible or detectable change as a result of reaction with AOS. Exposure may be directly within the body of the plant or crop or indirectly to a sample or an extract of the subcutaneous plant or crop matter from the damage or disease site, for example to a sample such as tissue explant, core, slice or piece. The chemical may be supported on a suitable support, for example on a paper, strip, stick, membrane, film or the like or may be provided in suspension or a solution.

Preferred chemicals for use in a chemical assay are those of the formazan or tetrazolium classes of dyes or similar conjugated aromatic dyes undergoing radical reaction with associated colour or fluorescence change, for example as disclosed in and following the protocol of Sutherland, M. W and Learmonth, B. A, “The Tetrazolium Dyes MTS and XTT Provide New Quantitative Assays for Superoxide and Superoxide Dismutase”, Free Rad, Res., Vol. 27 (3), pp 283-289, the contents of which are incorporated herein by reference.

Preferably chemical assay is with a formazan dye or tetrazolium dye more preferably the monotetrazolium dye, XTT (2,3-bis(2-methoxy-4-nitro-5-sulphophenyl)-5[(phenylamino)carbonyl]-2H-tetrazolium hydroxide, sodium salt), or MTS (5-(3-carboxymethoxyphenyl)-2-(4,5,-dimethylthiazolyl)-3-(4-sulphophenyl)tetrazolium, inner salt), a derivative of MTT (3- (4,5,-dimethylthiazol-2-yl)-2,5-diphenyl tetrazolium), or any of the WST family of tetrazolium compounds, preferably WST-1 or WST-8 (generic name water soluble tetrazoliums) as produced by Dojindo Laboratories, Tokyo, Japan. MTS generates a charged, water-soluble formazan when reduced, and MTS is more stable than XTT in the presence of phenazine methosulphate (PMS), a redox intermediate required in both assays. All reagents satisfy the criteria for quantitative .O₂⁻ assay.

The result of chemical assay is quantified by measurement of intensity of colour compared against a solution blank. A suitable means for quantification of chemical assay by intensity measurement is by spectrophotometry using the method of Sutherland and Learmonth (see above) the contents of which are herein incorporated by reference.

Preferably therefore the method comprises illuminating the exposed plant or crop, preferably a sample of excised tissue thereof, with light at a particular wavelength and measuring intensity of transmitted light, comparing readings for a sample including excised tissue and chemical assay reagent against a solution blank or against a control sample containing undamaged tissue and chemical assay reagent.

We have surprisingly found that the high levels of AOS produced in the biphasic response can be further emphasised by measuring calorimetric change at elevated pH. Preferably therefore assay is carried out at pH in the range 7 to 13, more preferably in the range 10 to 13.

We have moreover surprisingly found that the high levels of AOS produced in the biphasic response can be further emphasised by incubating for a period prior to measuring colorimetric change. Preferably therefore a sample for assay is incubated for 3 to 15 minutes prior to illuminating and detecting transmitted light.

We have moreover surprisingly found that the high levels of AOS produced in the biphasic response can be further emphasised by detecting colorimetric change under light at elevated wavelength. Preferably therefore spectrophotometry is carried out at wave length in the range lamda=450 nm to 650 nm, for example lamda=450 nm or 555 nm or 600 nm.

Electro chemical assay is for example by contacting the plant or crop to be assayed an electronic probe or sensor which maintains a potential difference across a membrane or the like, between a reference electrode and a test electrode, wherein the test electrode is capable of binding, absorption or otherwise interacting with AOS, thereby causing a change in potential and producing a change in potential difference which may be amplified. Contacting may be directly within the body of the plant or crop or indirectly to a sample or an extract of the subcutaneous plant or crop matter from the damage or disease site, for example to a sample such as tissue explant, core, slice or piece.

Electro-chemical assay techniques are disclosed including cyclic voltammetry, on voltammetric electrodes modified by means of mediators and suitable promoters (L. Campanella, G. Favero, M. Tomassetti, Sens. Actuators, B 44 (1997) 559-565), potentiometric sensors and polymeric membrane IS-FETs rendered selective by “spin trapping” reaction triggered by a specially synthesised specific nitrone contained in a PVC and sebecate membrane (L. Campanella, G. Favero, F. Occhionero, M. Tomassetti, Analysis 26 (1998) 223-228. L. Campanella, G. Favero, F. Occhionero, M. Tomassetti, in: C. Di Natale, A. D'amico, S. Sberveglieri (Eds.), Sensors and Microsystem, Proceedings of the 3^{rd} Italian Conference, Genova, Italy, 11-13 Feb. 1998, World Scientific, Singapore, 1999, pp. 219-224.) and more recently by means of bio-sensor techniques, for example by superoxide dismutase (SOD) enzymatic sensors, in which the enzyme is coupled to a gaseous diffusion electrode for oxygen or to an amperomotric electrode for hydrogen peroxide (L. Campanella, G. Favero, M. Tomassetti, Anal. Lett. 32 (1999) 2559-2581.).

Preferably electro chemical assay is by means of bio-sensor, using a modification of the protocol (L. Campanella, L. Persi, M. Tomassetti “A New Tool for Superoxide and Nitric Oxide Radicals Determination Using Suitable Enzymatic Sensors”, Sensors and Actuators B68 (2000) 351-359), in which the response is inhibited or enhanced (i.e. modulated) by the presence of superoxide or a nitric oxide radical, the contents of which are incorporated herein by reference.

Preferably a bio-sensor comprises enzymes supported on a membrane or similar film as hereinbefore defined between respective electrodes, for example cross-linked onto the membrane, absorbed, adsorbed or otherwise insolubilised. Preferably enzymes are selected from tyrosinase, superoxide dismutase, galactose oxidase and cytochrome oxidase.

The method of the invention employing exposing to direct damage or disease is typically a destructive method, moreover quantitative assay of AOS requires subcutaneous penetration into the tissue of the plant or crop, however since all plants and crops are typically harvested and stored and sold in bulk the method is suitable for detecting response in a sample of one or more items from a given harvest or storage, for assay, the result of which is representative of the harvest or storage stock in general. In a particular advantage however the method of the invention employing exposing to indirect damage or disease, preferably exposing to a damaged tissue extract, is non destructive, simply eliciting the bruise response, and may be used with non destructive assay, for plants or crops if desired.

However we have found that synthesis of AOS is highly localised and specific to the site of damage or disease. Preferably therefore the method comprises detecting AOS in a quantitative assay of subcutaneous plant or crop tissue which is coincident with or local to a suspected or induced site of damage or disease. Preferably assay is conducted at the core site of exposure to disease or damage rather than at the periphery, whereby the location of maximum AOS synthesis is assayed.

Preferably assay is at a preferred site in any given plant or crop. In the case of potato tubers, assay is at the stolon end (which was attached to the plant) and control is at the eye-end (end characterised by shoot (bud) endings not attached to plant). In the case of potato tubers preferably tissue is assayed or excised for assay from two mm out with the vascular ring, the vascular ring being a visible ring of tissue containing the xylem and phloem vessels occurring 1-10 mm from the epidermis of the tuber.

Preferably the quantitative assay for AOS comprises inserting assay means subcutaneously into a site which has been subject to or is suspected of damage or disease, or excising a sample of tissue from the site for contact with the assay means. Preferably contact with assay means is for a suitable period to achieve a chemical reaction, in the case of a chemical assay or to achieve radical reaction in an electro chemical assay.

It is a particular advantage of the invention that assays may be quantitative since intensity of bruising has been found to correspond to concentration of AOS detected, moreover has been found to be characterised by a concentration profile with time having a maximum at first and second times t as hereinbefore defined. If it is desired to obtain a quantitative indication of intensity of bruising, multiple assays may be conducted over a time period within the range t=0-7 hours.

The method of the invention may be employed with any plant or crop which is susceptible to bruise or discolouration due to mechanical damage or disease, including vegetable, fruit and plant products. The method may be used to detect response of the plant or crop during growth or during or subsequent to harvesting and it is particular envisaged in the present invention to employ the method at any stage during the growth or harvesting cycle as an indicator of the potential response of the plant or crop product once harvested, or of the harvested plant or crop product. Preferably therefore the method comprises in a precursor stage selecting a plant or crop to be sampled in vivo during growth or once harvested or during storage.

The following is a non exclusive list of suitable vegetable, fruit and plant products for which response to damage or disease would be detected accordingly to the method of the invention:

Potato (seed, processor or consumer), apple, lychee, pear, apricot, peach, orange (juice), banana, leaf or stem browning in cut flowers, lettuce, pomegranate, grape, mushroom, logan fruit, loquat fruit, blueberry, carambola, dog rose, rambutan, coconut, avocado, plantain, pineapple, walnut, artichoke, sugarbeet and onion.

Preferably the method is for detecting response in potato, apple, pear, leaf or stem browning in cut flowers, lettuce, mushroom and sugarbeet, most preferably in potato tuber, sugarbeet or apple.

In a further aspect of the invention there is provided an apparatus for detecting response to damage or disease in a plant or crop, by means of detecting AOS in a quantitative assay. Preferably the apparatus of the invention is an apparatus for diagnosing susceptibility to response.

In a first embodiment the apparatus of the invention comprises means for exposing a sample plant or crop to direct or indirect damage or disease and means for excising a sample from the exposed site for subsequent assay for AOS.

In an alternative embodiment the apparatus of the invention comprises means for exposing a sample plant or crop to direct or indirect damage or disease and means for detecting AOS generated as a result of exposure to damage or disease, in a quantitative assay.

The apparatus of the invention may comprise or be used with any known assay means, preferably for chemical or electro chemical assay, in the form of a support or reservoir for a solid, solution or suspension chemical for assay, a probe or sensor for electro chemical assay and the like.

As hereinbefore defined the synthesis of AOS is highly specific and local to the site of exposure to damage or disease and it is possible to achieve a false result by assaying the plant or crop at a site other than the main site of exposure. It is a particular advantage of the present invention that a combined apparatus having both damage or disease exposing means and sampling or assay means is able to accurately pinpoint a site for exposure and locate this site for sampling or for in situ assay.

An apparatus of the invention for exposing to direct o indirect damage or disease comprises a suitable impactor to induce damage; or means for subcutaneously introducing damaged or diseased tissue or extract thereof such as disease substances or organisms or damaged tuber extracts, for example by a contaminated advancing blade, advancing syringe and the like; or comprises a reservoir or cell for damaged or diseased tissue or extract thereof, having a membrane or permeable site for release or contact of reservoir or cell contents.

Preferably the impactor intensity may be varied as desired. Preferably the advance of blade or syringe may be varied as desired. The contact site of a reservoir or cell may include means to pierce the plant or crop sample and achieve subcutaneous impregnation. Alternatively the extract may include digesting means to digest the plant or crop surface.

Preferably the apparatus of the invention comprises a contact face which is adapted to be located against the desired site of the plant or crop to be damaged and which corresponds with the surface area of the site to be exposed to damage or disease. Preferably the contact face includes means to mark the site, for example by colouration, indentation or incision marks, or includes a sampling means comprising a corer element for incising a core sample of the plant or crop and removing. The apparatus is suited for use with means for recording time delay t from exposing to damage or disease prior to assay.

The apparatus may as hereinbefore defined comprise separate or integral means for subsequent assay. In the case that assay means is separate a further conventional assay kit is then applied to the marked or cored site. In the case that assay means is integral, an impactor, incisor or injector, or reservoir comprises access means for providing access for a chemical support or probe or sensor to penetrate the impacted, injected or incised or otherwise exposed region of the plant or crop to a pre-determined depth within the damage or diseased site and contact the plant or crop matter at that site. Preferably assay means comprises an advancing probe having chemical absorbed on paper or in a reservoir, or electrodes forming part of a sensor, for direct contact of paper or sensor or membrane or similar diffusion contact of chemical substance with the plant or crop matter. Advancing means for chemical assay may be a blade or other divider which splits the impact or injection site for the purpose of inserting the chemical therebetween.

Control detection of AOS may be with use of a second apparatus of the invention or of second assay means at a control non-exposed site or on a control non-exposed plant or crop. In the case of chemical assay comparison of response may be by visual comparison or by measuring intensity of light transmission or by light emission (fluorescence) or the like. The apparatus may comprise any suitable calibration and intensity measuring device known in the art. Preferably the apparatus comprises communication means to communicate between respective calibration and intensity detecting means, whereby these are calibrated for local chemical concentration and subsequently a single response is displayed comprising the difference in AOS and level at the exposure site and the control site.

In the case of an electro chemical assay, the apparatus of the invention preferably comprises means for communicating between an assay sensor and a control sensor together with means for comparison of the respective signals, either in the form of circuitry serving to subtract one signal from the other and to produce a single result, or serving to reverse the polarity of one sensor with respect to the other, thereby subtracting the signals and producing a net result.

It is a particular advantage of the invention that an apparatus as hereinbefore defined is suited for the rapid analysis of small or large numbers of samples and may be used in assessing the status of a cross section of a crop field or store. The apparatus of the invention may be provided in configuration or capacity suited for a different crop or plant types as hereinbefore defined, in particular may be provided in different sizes, with different ranges of impactor level, for example to impart greater energy in the region of 0.7 joules for hard plants or crops such as potatoes, and for plants and crops having an outer dermis such as bananas, and having a lesser energy for soft plants and crops such as mushroom and the like.

In a further aspect of the invention there is provided a kit for diagnosing susceptibility to response to damage or disease as hereinbefore defined comprising an apparatus as hereinbefore defined having separate or integral assay means, and separate or integral control assay means, tissue extract reagents, assay reagents and buffers as required.

In a further aspect of the invention there is provided the use of an apparatus as hereinbefore defined in detecting a response of a plant or crop to damage or disease as hereinbefore defined.

In a further aspect of the invention there is provided a method for modifying the variety, growth or environmental conditions of a plant or crop which has been shown to produce a deleterious response to damage or disease. Susceptibility to damage or disease within a susceptible variety may be growth or environmental condition dependent, for example dependent on potassium nutrition, crop maturity, planting times and the like. Preferably favourable growth or environmental conditions are determined and applied to a susceptible variety to reduce susceptibility thereby optimising crop yield. Alternatively the variety is determined as being of enhanced susceptibility to damage or disease, whereby harvesting or storage conditions are modified to minimise impact or stress damage during harvesting and storage.

In a further aspect of the invention there is provided a method for selecting a variety which is resistant to damage or disease to an extent which is suitable for pre-determined growth or environmental or harvesting conditions.

In a further aspect of the invention there is provided a computer programmed with data relating to susceptibility of a plurality of plants and crops to damage or disease, derived by the method or with use of the apparatus or kit of the invention as hereinbefore defined, to provide a database for use in selection of a plant or crop for growth, handling or storage under a particular set of conditions, or to select optimum conditions for growth, storage or handling of a particular plant or crop.

The invention is now illustrated in non-limiting manner with reference to the following examples and Figures.

In the Figures:

FIG. 1 illustrates a scheme of the possible interrelations of Molecular Oxygen Species and Active Oxygen Species.

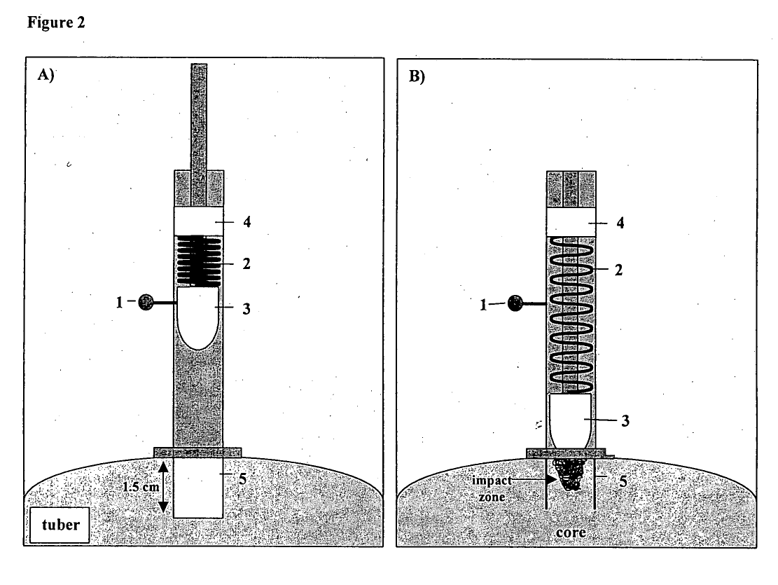

FIG. 2 illustrates a disposable apparatus of the invention comprising integral damage inducing means and sampling means. The apparatus comprises a spring loaded impact gun having trigger (1) to activate spring (2) supporting hammer (3). Spring-loaded hammer is adjustable and calibrated to impose a reproducible force, for example of about 0.7 joules (equivalent to the falling bolt and pendulum impact systems). Tension adjuster (4) is shown used to adapt impact intensity according to plant or crop or variety/cultivar.

The apparatus is designed to impose a controlled defined impact to the surface of test potato tubers and to precisely locate the bruise zone for subsequent assay. Positioning ring (5) is a circular cutter or corer which positions the impactor on the surface of the tuber and at the same time cuts a core of precise dimensions containing the tuber tissue sample. The Figure shows A) gun positioned and cocked and B) gun after firing showing the impact zone within the core.

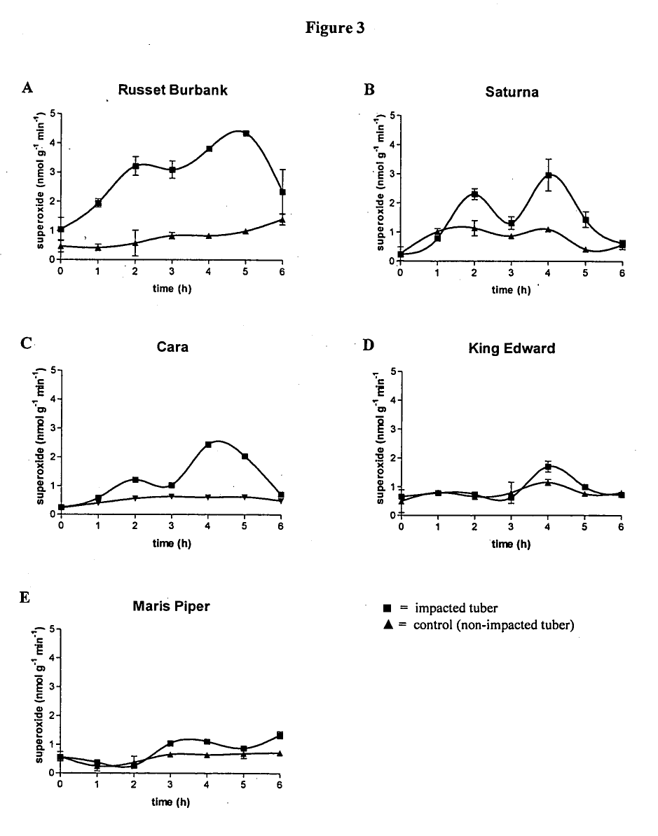

FIGS. 3(A-E) illustrates the time profile of superoxide radical assay in potato tubers. Time course (hours post-impact) and levels of superoxide (nmol g⁻¹ min⁻¹) generated in tubers exposed to mechanical stress. The five cultivars tested were A) Russet Burbank; B) Saturna; C) Cara; D) King Edward; E) Maris Piper. (▪-▪ mechanically stressed tissue, ▴-▴ control tissue). The results shown are the mean values for two independent estimates.

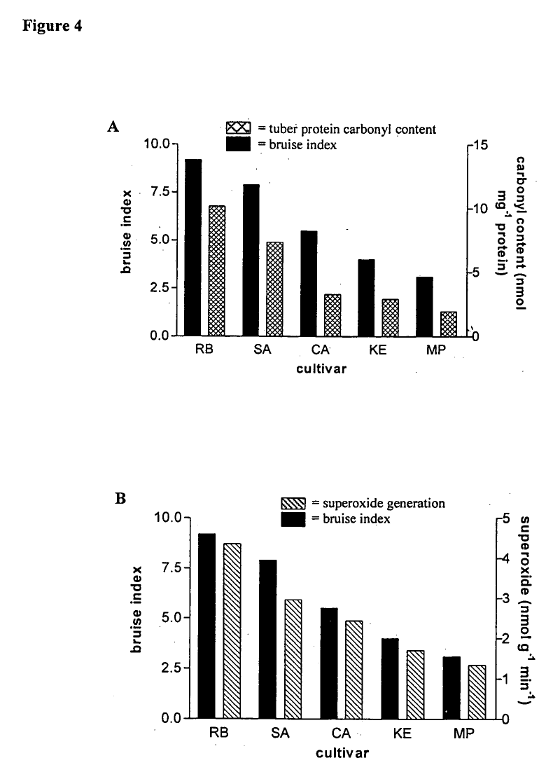

FIGS. 4(A, B) illustrates Histogram comparisons of cultivar bruise indices and levels of protein modification (Table 1) and maximum levels of superoxide generated following mechanical stress (FIG. 3). Results shown are the mean values for two independent estimates corrected for control levels and provides a visual representation of the correlations between A) bruise index (solid bars) and protein modification (cross hatched bars) and B) bruise index (solid bars) and superoxide production (cross hatched bars). Cultivar abbreviations: RB=Russet Burbank, SA=Saturna, CA=Cara, KE=King Edward, MP=Maris piper.

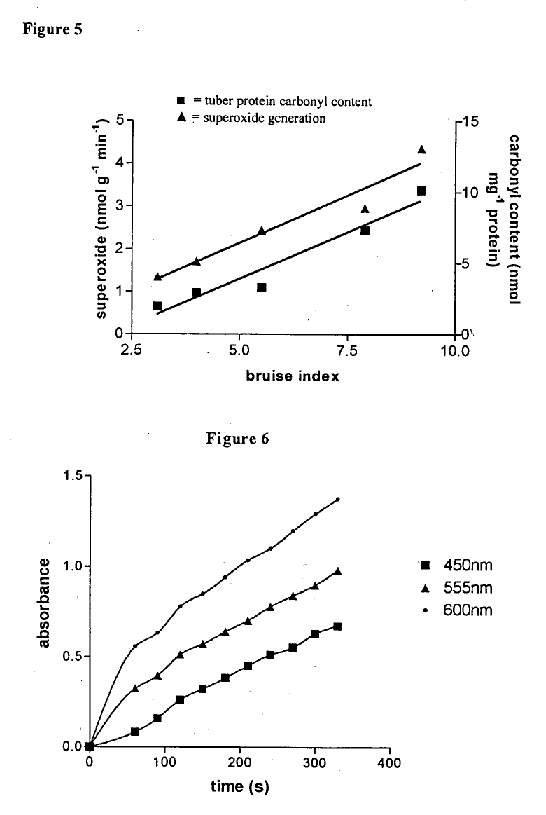

FIG. 5 illustrates correlation plots between bruise index values, protein modification and levels of superoxide generation. Graph shows the near straight line relationships between the levels of melanin pigment synthesis (bruise index) and both superoxide generation and protein modification. (▪-▪ protein modification, ▴-▴ superoxide generation).

FIG. 6 illustrates the enhanced detection sensitivity achieved by increasing the pH and measuring at wavelength 555 nm or 600 nm.

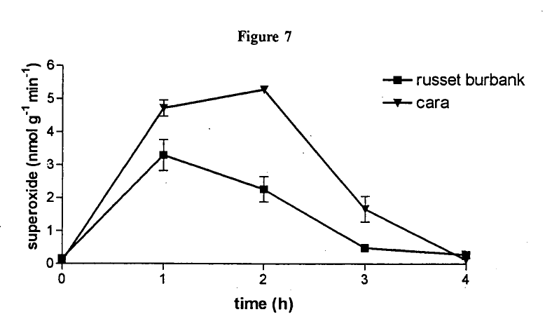

FIG. 7 illustrates the generation of AOS in excised tuber tissues following treatment with aqueous extracts taken from impacted tubers of cultivar Russet Burbank. Figure shows AOS generated in the same cultivar (Russet Burbank, ▪-▪) and a different cultivar (Cara, ▾-▾) in response to application of the extract.

### EXAMPLES

### Example 1

An XTT-based assay was used to measure directly superoxide radical production in tissues from mechanically stressed potato tubers. The results show that levels of superoxide synthesis vary between potato cultivars which also exhibit differing susceptibilities to blackspot bruising. This synthesis is biphasic with two peaks of superoxide accumulation over a 6 hour period. Changes in the level of protein modification in mechanically stressed tubers were also measured as postulated verification of susceptibilities to blackspot bruising. Visual inspection of bruising was also made as verification.

Materials

Tubers from selected potato cultivars, specifically grown and carefully harvested to avoid any mechanical stress, were kindly supplied by Dr Adrian Briddon (Sutton Bridge Experimental Unit, Sutton Bridge, Lincolnshire, UK). Other tuber materials were purchased locally from commercial outlets. Five potato cultivars were used—Cara, King Edward, Maris Piper, Russet Burbank and Saturna. All tubers were stored in the dark at 10° C. to inhibit greening and sprouting.

Tuber Mechanical Stress—Bruising. Tubers were washed in cold water and blotted dry, incubated for 48 h at 4° C. in the dark, returned to room temperature and then impacted at the stolon end using a standard falling weight of 240 g and a 300 mm drop height imparting a force of 0.7 J. The bolt was dropped through a plastic pipe to direct and contain impact, and the area of impact marked with an indelible marker. Impacted and control tubers were incubated for 48 h at 26° C. to allow maximal synthesis of bruise pigments.

Tuber Extracts—Where appropriate, extracts were prepared from tuber tissues exposed to a controlled impact. 5 mm³ slices of tuber tissue, excised using a clean sharp razor blade from the centre of impact sites (from 2 mm out with the vascular ring). The excised tissues were homogenised in 1 ml distilled water and centrifuged (15,000 g×5 min, room temperature) and the clarified soluble extract. 5 mm³ slices of non-impacted tuber tissue at the stolon end, were excised using a clean sharp razor blade, and exposed to 200 μl of the extract.

Assay of superoxide radical generation in tuber tissues: Tubers of each cultivar exposed to Mechanical Stress as described above were incubated in the dark at 26° C. for various periods of time prior to assay for superoxide generation. 5 mm³ slices of tuber tissue, excised using a clean sharp razor blade from the centre of impact sites (from 2 mm out with the vascular ring) and from distal control sites were washed thoroughly with distilled water (3×15 s), blotted dry and then incubated at 16° C. for 20 minutes in 200 μl 0.6 mM XTT in 50 mM phosphate buffer, pH 8.2 (Able et al. 1998). The tissue pieces were removed with forceps and the assay solution centrifuged (13000 g) for 5 minutes at room temperature. The A₄₅₀ of the supernatant was taken on a 100 microlitre sample in a spectrophotometer (lamda=450 nm) comparing against a XTT solution blank (200 microlitre of 600 micromolar XTT in 50 mM phosphate buffer pH8.2) and converted to μmol superoxide generated using the molar extinction coefficient for the XTT formazan of 23,600 M⁻¹ cm⁻¹ (Sutherland and Learmonth, 1997; Sutherland M, personal communication). The solution was adjusted to pH 12.5 using 10M NaOH and read at 555 nm. The solution was then incubated at pH 12.5 for 10 minutes and the wavelength read at 600 nm. Superoxide estimations were carried out in duplicate and all assays were replicated.

The results are shown in FIGS. 3(A-E) which show the time course of superoxide generation in tuber tissue slices. All cultivars showed some degree of superoxide production in response to impact. Very significant levels were produced by cv. Russet Burbank and cv. Saturna (FIGS. 3-B) while cv. Maris Piper (FIGS. 3-E) displayed only slightly elevated levels compared with the control values. Most striking was the biphasic nature of the superoxide generation pattern over time with an initial peak response at 1-2 hours post-impact followed by a larger response at 4-5 hours. This pattern was reproducible and apparent in all five cultivars although most obvious in cv. Russet Burbank, cv. Saturna, and cv. Cara (FIGS. 3A-C) which also showed the highest bruise indices. The peak values for superoxide generation were plotted against bruise index for the five cultivars to illustrate the relationship between oxidative burst and melanin pigment synthesis (FIG. 4B). The enhanced sensitivity of the method by increasing the pH as described is shown in FIG. 6. The induction of AOS generation by exposure of non-impacted tuber tissues to an extract prepared from impacted tuber tissues is shown in FIG. 7. Preparation of the whole or part tuber extracts which activate AOS generation in a cultivar-specific manner would supplement the means to diagnose susceptibility to bruising, in particular by gains in sensitivity and speed of detection.

Estimation of protein modification in tuber tissues: The carbonyl content of oxidatively modified tuber proteins was quantified using an OxyBlot kit (Oncor Appligene) by the dinitrophenylhydrazine (DNPH) derivatisation and spectrophotometric assay method of Levine R L, “Mixed function oxidation or histidine residues”, Methods in Entymology, 107, 370-376 (1984). Tubers exposed to Mechanical Stress by the method of the invention as described above were incubated for 48 h. Tuber proteins were extracted from 150mg of control and impacted tubers in 3 ml of 50 mM phosphate buffer, pH7.4. The carbonyl groups on extracted proteins were reacted with DNPH and the resulting hydrazone derivatives estimated from the peak absorbance at 355-390 nm using a molar extinction coefficient of 22,000 M⁻¹ cm⁻¹. Protein contents of duplicate samples were estimated from the A₂₈₀. Values were expressed as nmol carbonyl mg⁻¹ tuber protein.

The results are shown in Table 1 and FIG. 4A and FIG. 5 correlated against superoxide production and visual bruise index.

Assessment of Bruising—Visual Inspection Tubers were exposed to Mechanical Stress as described above and incubated and were then cut in quarters at the impact site and the volume of affected tissue measured as well as a visual estimation of the intensity of pigmentation on a scale of 0 (no discoloration) to 10 (deep blue-black coloration). 30 tubers for each of the five cultivars were used and a mean bruise index calculated based on bruise extent and intensity. The results are shown in Table 1.

| | Carbonyl Content (nmol | |
|---|---|---|
| Cultivar | mg⁻¹ tuber protein) | Bruise Index |
| Russet Burbank | 10.1 | 9.2 |
| Saturna | 7.4 | 7.9 |
| Cara | 3.3 | 5.5 |
| King Edward | 2.9 | 4.0 |
| Maris Piper | 2.0 | 3.1 |

Table 1—Bruise index (visual estimation and protein oxidative modification) of potato cultivars: Table 1 lists the values estimated for the bruise susceptibilities for each of the five experimental cultivars. The cultivars used exhibited a wide range of susceptibilities to bruise development from highly susceptible (cv Russet Burbank) to highly resistant (cv Maris Piper). Our estimates of bruise indices for the cultivars used here are largely comparable with published values using similar methods (Dixon, 1992; British Potato Council, 2000).

The results show that protein modification values (FIG. 3A) closely mirror those for the levels of superoxide generation (FIG. 3B) in that cv. Russet Burbank showed the highest level of modified protein while cv. Maris Piper had the lowest. Furthermore comparison of the bruise indices for the cultivars (Table 1) with levels of protein modification (FIG. 3A) and with superoxide generation (FIG. 3B) show that there is a direct relationship between these variables. Thus high superoxide generation leads to high protein modification and both are linked to high bruise susceptibility.

1. Method for detecting response to damage in a potato plant or crop comprising detecting Active Oxygen Species (AOS) selected from superoxide (.O₂⁻), nitric oxide radical NO., peroxynitrite radical ONOO⁻ and hydroxyl radical .OH, in a quantitative assay in the first or second peak concentration of AOS of a biphasic response, at a time t from damage, in which the response in terms of AOS production is linear to the bruising response.

2. Method for detecting response to disease in a potato plant or crop comprising detecting Active Oxygen Species (AOS) selected from superoxide (.O₂⁻), nitric oxide radical NO., peroxynitrite radical ONOO⁻ and hydroxyl radical .OH, in a quantitative assay in the first or second peak concentration of AOS of a biphasic response, at a time t from damage, in which the response in terms of AOS production is linear to the bruising response.

3. Method as claimed in any of claims 1 or 2 wherein Active Oxygen Species comprises superoxide radical (.O₂⁻).

4. Method as claimed in any of claims 1 to 3 which comprises detecting AOS generated and comparing to indicate damage or disease status, wherein quantitative assay is chemical or electrical and gives a colour or electric signal response which is graded by intensity of colour or magnitude of electronic signal.

5. Method as claimed in any of claims 1 to 4 wherein time t is 0.5 to 2 hours or 4.5 to 6 hours after exposure.

6. Method as claimed in any of claims 1 to 5 which comprises detecting AOS in a second peak of bi-phasic response.

7. Method as claimed in any of claims 1 to 6 which is a method for detecting whether damage or disease has occurred, and comprises conducting the assay on the suspected site of damage or disease.

8. Method as claimed in any of claims 1 to 6 which is a method for diagnosing the susceptibility of a potato tuber from the plant or crop to respond to damage or disease, and comprises in a first stage exposing a potato tuber from the plant or crop to damage or disease and in a second stage detecting AOS in a quantitative assay, at a time t after exposure.

9. Method as claimed in any of claims 1 to 8 wherein exposure is deliberate or incidental and is directly within the body of a potato tuber from the plant or crop or indirectly to a sample comprising tissue explant, core, slice or piece of the subcutaneous matter from the damage or disease site of a potato tuber from the plant or crop.

10. Method as claimed in any of claims 1 to 9 wherein exposure to damage or disease is direct and comprises deliberate or incidental exposure to mechanical impact, pressure, vibration or shock or exposure to disease substances, organisms, fungus, bacteria or virus by contact, injection or otherwise contamination.

11. Method as claimed in claim 10 wherein mechanical impact is deliberate and is a representative controlled impact energy.

12. Method as claimed in claims 1 to 11 wherein exposure to damage or disease is indirect and comprises exposure to damaged or diseased tissue or components thereof, or subjecting to conditions inducing tissue or cell damage or disease such as swelling, humidity, freezing and the like.

13. Method as claimed in claim 12 wherein damaged or diseased tissue is of the same or different potato variety, and is preferably a different variety.

14. Method as claimed in any of claims 10 to 13 wherein the stage of inducing damage directly in a potato tuber from the plant or crop or preparing damaged tissue for extract and indirect exposure of a potato tuber from the plant or crop comprises subjecting the tuber to impact at the site to be assayed as hereinbefore defined at intensity of impact in the energy (or work) range of 0.5 to 1.0 Joules.

15. Method as claimed in any of claims 12 to 14 wherein the stage of indirectly exposing to damage comprises contacting with part or the whole of an extract prepared from impacted tissue.

16. Method as claimed in claim 15 wherein an extract from tissues exposed to impact comprises excised tissues homogenised and centrifuged to give a clarified soluble extract, optionally further treated to isolate a particular component of the extract or to add additional components such as diffusion enhancers, skin digestants and the like.

17. Method as claimed in any of claims 15 and 16 wherein an extract comprises synthetically prepared equivalents of the damage tissue extract or derivatives or analogues thereof.

18. Method as claimed in any of claims 4 to 18 wherein chemical assay is by exposing to a potato tuber of the plant or crop to be assayed a chemical marker which is known to undergo visible or detectable change as a result of reaction with AOS.

19. Method as claimed in any of claims 4 to 18 wherein chemicals for use in a chemical assay include those of the tetrazolium class of dyes, preferably monotetrazolium dye which generates a charged, water-soluble formazan when reduced.

20. Method as claimed in any of claims 18 to 20 wherein quantification of chemical assay by intensity measurement is by spectrophotometry.

21. Method as claimed in claim 18 to 20 wherein a sample for assay is incubated for a period and assay is carried out at pH in the range 7 to 13, more preferably in the range 10 to 13.

22. Method as claimed in claim 20 wherein spectrophotometry is carried out at wave length in the range lamda=450 nm to 650 nm.

23. A diagnostic reagent for use in the Method of any of claims 1 to 22 comprising an extract of damaged or diseased tissue of a potato tuber, or active component thereof, or synthetic equivalent or derivative thereof, optionally comprising additional components useful for aiding penetration or contact with a sample to be diagnosed wherein the extract comprises a natural or synthetic extract of the same or different potato plant or crop to that to be diagnosed.

24. Diagnostic reagent as claimed in claim 23 which comprises extract or component(s) in effective diagnostic amount.

25. Diagnostic reagent as claimed in claim 23 or 24 wherein the extract is of a different potato plant or crop which is found to have high diagnostic value.

26. Apparatus for detecting response to damage in a potato plant or crop; using the method as hereinbefore defined in any of claims 1 to 22; wherein the apparatus comprises an impactor to impose a controlled impact energy in the range 0.5 to 1.0 Joules to induce damage and to precisely locate the impact site for subsequent assay.

27. Apparatus as claimed in claim 26 which comprises means for excising a sample from the exposed site for subsequent assay for AOS.

28. Use of a method, reagent or apparatus as defined in any of claims 1 to 27 in detecting response to damage or disease in a potato plant or crop.

29. Kit for diagnosing susceptibility to response to damage or disease in a potato plant or crop using the method as hereinbefore defined in any of claims 1 to 22 comprising an apparatus as hereinbefore defined in claim 26, separate or integral chemical assay means, and/or a diagnostic reagent as hereinbefore defined in any of claims 30 to 32, tissue extract reagents, assay reagents and buffers as required and means for colour grading a colour response from a chemical assay as hereinbefore defined in claim 4.

30. Method for modifying the growth or environmental conditions of a potato plant or crop which has been shown to produce a deleterious response to damage or disease by a method as hereinbefore defined in any of claims 1 to 22.

31. Method for selecting a variety of potato plant or crop which has been determined as resistant to damage or disease by a method as hereinbefore defined in any of claims 1 to 22 to an extent which is suitable for pre-determined growth or environmental or harvesting conditions.
